(19) United States
(12) Patent Application Publication
(10) Pub. No.: US 2021/0186402 A1
(43) Pub. Date: Jun. 24, 2021

(54) ELECTROCARDIOGRAM MEASURING DEVICE

(71) Applicant: Intelliances Incorporation, New Taipei City (TW)

(72) Inventors: Ho-Chi Fu, New Taipei City (TW); Chun-Yu Chen, New Taipei City (TW)

(73) Assignee: Intelliances Incorporation, New Taipei City (TW)

(21) Appl. No.: 17/126,044

(22) Filed: Dec. 18, 2020

(30) Foreign Application Priority Data
Dec. 24, 2019 (TW) 108217130

(51) Int. Cl. A61B 5/282; A61B 5/00; A61B 5/335

(52) U.S. Cl. CPC A61B 5/282; A61B 5/0006; A61B 2562/0219; A61B 2560/0214; A61B 5/335

(57) ABSTRACT

An electrocardiogram measuring device, including a first machine body, a second machine body, a third machine body, a circuit module, and two connecting members, is provided. The first machine body, the second machine body, and the third machine body respectively has an exposed first electrode, an exposed second electrode, and an exposed third electrode. The circuit module is disposed in one of the three machine bodies. The first electrode, the second electrode, and the third electrode are connected to the circuit module via an electrical signal. One of the connecting members is located between and connected to the first machine body and the second machine body. The other connecting member is located between and connected to the first machine body and the third machine body. A non-zero included angle is formed between the two connecting members. Relative positions of the three machine bodies are fixed through the two connecting members.

### CROSS-REFERENCE TO RELATED APPLICATION

This application claims the priority benefit of Taiwan application serial no. 108217130, filed on Dec. 24, 2019. The entirety of the above-mentioned patent application is hereby incorporated by reference herein and made a part of this specification.

### BACKGROUND

### Technical Field

This disclosure an electrocardiogram measuring device, and in particular to an electrocardiogram measuring device with a fool-proof function.

### Description of Related Art

The heart is composed of muscles capable of spontaneous beating and rhythmic contraction. The activity of the myocardium is dominated by electrical current generated by the sinoatrial node and the atrioventricular node. The current will also be reflected to the body surface through conductive tissues and body fluids around the heart. An electrocardiogram (ECG) is a graphic in which changes in voltage of the heart tissues are recorded using microelectrode technology.

Currently, most people have to go to the hospital if they wish to measure the ECG, where trained professionals will operate an ECG apparatus to obtain the ECG results. An ECG equipment provided for the general public or an untrained personnel to measure the ECG on their own is relatively rare. This is because during the measurement of the ECG, multiple electrodes have to be placed on the body and the measurement results may be affected because of wrong placement of the electrodes by the untrained personnel.

### SUMMARY

This disclosure provides an electrocardiogram measuring device, which has a fool-proof function and can be operated by a general user.

An electrocardiogram measuring device according to the disclosure includes a first machine body, a second machine body, a third machine body, a circuit module, and two connecting members. The first machine body has an exposed first electrode. The second machine body has an exposed second electrode. The third machine body has an exposed third electrode. The circuit module is disposed in one of the first machine body, the second machine body, and the third machine body. The first electrode, the second electrode, and the third electrode are connected to the circuit module via an electrical signal. One of the connecting members is located between the first machine body and the second machine body, and connected to the first machine body and the second machine body. The other connecting member is located between the first machine body and the third machine body, and connected to the first machine body and the third machine body. A non-zero included angle is formed between the two connecting members. Relative positions of the first machine body, the second machine body, and the third machine body are fixed through the two connecting members.

In an embodiment of the disclosure, the two connecting members are two transmission lines, and the first machine body, the second machine body, and the third machine body are electrically connected to each other through the two transmission lines.

In an embodiment of the disclosure, the non-zero included angle formed between the two connecting members is between 60 degrees and 120 degrees.

In an embodiment of the disclosure, the non-zero included angle formed between the two connecting members is 90 degrees.

In an embodiment of the disclosure, the two connecting members have a same length, which enables the second machine body and the third machine body to be symmetrically disposed on two sides of the first machine body.

In an embodiment of the disclosure, the electrocardiogram measuring device further includes at least one battery, which is disposed in at least one of the first machine body, the second machine body, and the third machine body.

In an embodiment of the disclosure, the at least one battery includes two batteries, which are disposed in the second machine body and the third machine body, and the circuit module is disposed in the first machine body.

In an embodiment of the disclosure, the circuit module includes a processor and a Bluetooth chip electrically connected to the processor, and the first electrode, the second electrode, and the third electrode are electrically connected to the processor.

In an embodiment of the disclosure, the circuit module includes a processor and a gyroscope electrically connected to the processor, and the first electrode, the second electrode, and the third electrode are electrically connected to the processor.

In an embodiment of the disclosure, the circuit module includes a processor and an artificial intelligence chip electrically connected to the processor, and the first electrode, the second electrode, and the third electrode are electrically connected to the processor.

In an embodiment of the disclosure, the length, width or diameter of any one of the first machine body, the second machine body, and the third machine body are respectively between 2 cm and 8 cm, and the length of any one of the two connecting members is between 2 cm and 8 cm.

Based on the above, the electrocardiogram measuring device according to the disclosure uses the two connecting members to fix the relative positions of the first machine body, the second machine body and the third machine body. The relative positions of the first electrode, the second electrode and the third electrode are fixed correspondingly too. Compared with the conventional electrocardiogram, in which the operator has to place the electrodes one by one, and the relative positions of the electrodes may be wrong, causing the measurement result to be distorted, the electrocardiogram measuring device according to the disclosure provides an alternative that can be operated by the general user by himself/herself, while being quite convenient to use.

To make the above mentioned features and advantages of the disclosure more comprehensible, exemplary embodiments in concert with drawings are described in detail below.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and together with the description, serve to explain the principles of the disclosure.

FIG. 1 is a schematic diagram of an electrocardiogram measuring device according to an embodiment of the disclosure.

FIG. 2 is a schematic diagram of the electrocardiogram measuring device in FIG. 1 during measurement.

FIG. 3 is a schematic diagram of an electrocardiogram measuring device according to another embodiment of the disclosure.

FIG. 4 is a schematic diagram of an electrocardiogram measuring device according to yet another embodiment of the disclosure.

### DESCRIPTION OF THE EMBODIMENTS

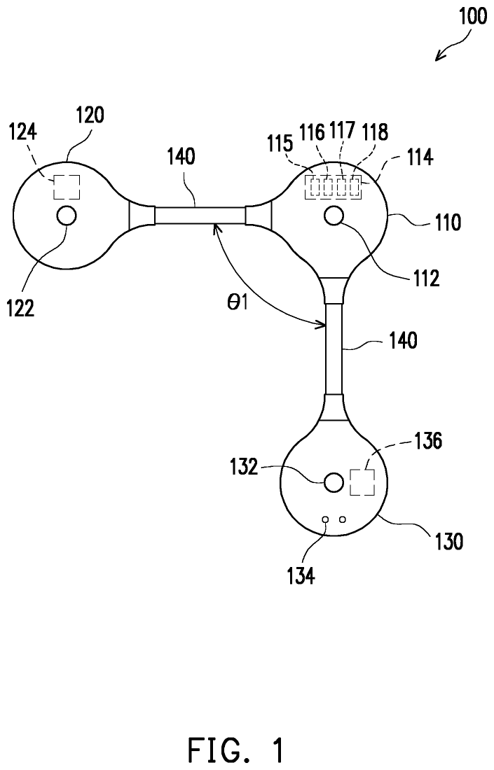

FIG. 1 is a schematic diagram of an electrocardiogram measuring device according to an embodiment of the disclosure. With reference to FIG. 1, an electrocardiogram measuring device 100 of the embodiment uses a six-lead electrocardiogram measuring device as an example. The electrocardiogram measuring device 100 of the embodiment includes a first machine body 110, a second machine body 120, and a third machine body, a circuit module 114 and two connecting members 140.

The first machine body 110 has an exposed first electrode 112, the second machine body 120 has an exposed second electrode 122, and the third machine body 130 has an exposed third electrode 132. The circuit module 114 is disposed in one of the first machine body 110, the second machine body 120, and the third machine body 130. In the embodiment, the circuit module 114 is disposed in the first machine body 110, but in other embodiments, the circuit module 114 may be disposed in the second machine body 120 or the third machine body 130. The disposition position of the circuit module 114 is not limited by the drawings.

As can be seen from FIG. 1, one of the connecting members 140 is located between the first machine body 110 and the second machine body 120, and is connected to the first machine body 110 and the second machine body 120. The other connecting member 140 is located between the first machine body 110 and the third machine body 130, and is connected to the first machine body 110 and the third machine body 130. A non-zero included angle θ1 is formed between the two connecting members 140. In the embodiment, the non-zero included angle θ1 between the two connecting members 140 uses an example of 90 degrees, but it is not limited thereto.

Compared with the conventional electrocardiogram, in which the operator has to place the electrodes one by one, and relative positions of the electrodes may be wrong, causing the measurement result to be distorted, the first machine body 110, the second machine body 120 and the third machine body 130 fix their relative positions through the two connecting members 140. The relative positions of the first electrode 112, the second electrode 122 and the third electrode 132 are fixed correspondingly too.

Since the relative positions of the first electrode 112, the second electrode 122, and the third electrode 132 of the electrocardiogram measuring device 100 are fixed, a user only has to place the electrocardiogram measuring device 100 on the chest during operation. More specifically, the user only has to make sure that the first machine body 110 is placed beside the left breast near to the armpit (a horizontal position level to the armpit), and the connecting member 140 connecting the first machine body 110 and the second machine body 120 is approximately horizontal, then the positions of the second machine body 120 and the third machine body 130 may be determined. Therefore, the electrocardiogram measuring device 100 of the embodiment can effectively eliminate the need for the user to place the electrodes one by one, therefore providing an alternative that can be operated by the general user by himself/herself, while being quite convenient to use.

It is worth mentioning that length, width or diameter of any one of the first machine body 110, the second machine body 120, and the third machine body 130 are respectively between 2 cm and 8 cm. Length of any one of the connecting members 140 is between 2 cm and 8 cm. Therefore, the electrocardiogram measuring device 100 has a small size, making it very convenient to carry around and easy to place on the body. In the embodiment, the first machine body 110, the second machine body 120, and the third machine body 130 are oblate shapes, but the shapes of the first machine body 110, the second machine body 120, and the third machine body 130 are not limited thereto, and may also be rectangular, polygonal or other shapes.

In the embodiment, the first electrode 112, the second electrode 122, and the third electrode 132 are connected to the circuit module 114 via an electrical signal. In other words, the signals measured by the first electrode 112, the second electrode 122, and the third electrode 132 are transmitted to the circuit module 114. In the embodiment, the two connecting members 140 are two transmission lines, and the first machine body 110, the second machine body 120, and the third machine body 130 are electrically connected to each other through the two transmission lines. Therefore, the signals measured by the second electrode 122 and the third electrode 132 may be transmitted to the circuit module 114 in the first machine body 110 by the two transmission lines.

Certainly, in other embodiments, the connecting member 140 may not have the transmission function, and only have the function of fixing the relative position. In such an embodiment, the first machine body 110, the second machine body 120, and the third machine body 130 may all be disposed with communication units to transfer messages in a wireless manner (such as Bluetooth).

In addition, in the embodiment, the connecting member 140 may be slightly flexible to provide convenience during storage. However, the connecting member 140 has to be slightly hard overall to facilitate fixing of the relative positions of the first machine body 110, the second machine body 120, and the third machine body 130. Certainly, in other embodiments, the connecting member 140 may also be rigid, and is not limited by the above.

In addition, in the embodiment, the two connecting members 140 are of equal length, and the second machine body 120 and the third machine body 130 are symmetrically disposed on two sides of the first machine body 110. Certainly, in other embodiments, the lengths of the two connecting members 140 may also be designed to be different according to requirements, and is not limited by the drawings.

On the other hand, to provide ease of use to the user, the electrocardiogram measuring device 100 further includes at least one battery, such as two batteries 124 and 136, which are disposed in at least one of the first machine body 110, the second machine body 120, and the third machine body 130, and electrically connected to the circuit module 114. The batteries 124 and 136 are configured to store power. Therefore, the user may directly use the electrocardiogram measuring device 100 after charging, without having to supply additional power through a power transmission line.

More specifically, in the embodiment, the two batteries 124 and 136 are disposed in the second machine body 120 and the third machine body 130. Since the second machine body 120 and the third machine body 130 are symmetrically disposed on the two sides of the first machine body 110, by disposing the two batteries 124, 136 in the second machine body 120 and the third machine body 130 may enable weight to be distributed uniformly.

Certainly, in other embodiments, the number of the batteries is not limited thereto, and a designer may adjust the number according to the requirements. It is also possible that only a single machine body is disposed with a battery or all three machine bodies are respectively disposed with a battery. In addition, it is also possible to dispose more than one battery in a single machine body. The disposition manner of the battery is not limited thereto.

In addition, the third machine body 130 has a charging contact point 134, which is electrically connected to the batteries 124 and 136. The electrocardiogram measuring device 100 may be electrically connected to an external charging stand (not shown) through the charging contact point 134. The third machine body 130 and the external charging stand may also be disposed with two magnetic elements (not shown, such as magnets) to facilitate quick alignment and fixation during charging.

The circuit module 114 further includes a processor 115, and the first electrode 112, the second electrode 122, and the third electrode 132 are electrically connected to the processor 115. In addition, the circuit module 114 may selectively include a Bluetooth chip 116, a gyroscope 117, or/and an artificial intelligence (AI) chip electrically connected to the processor 115.

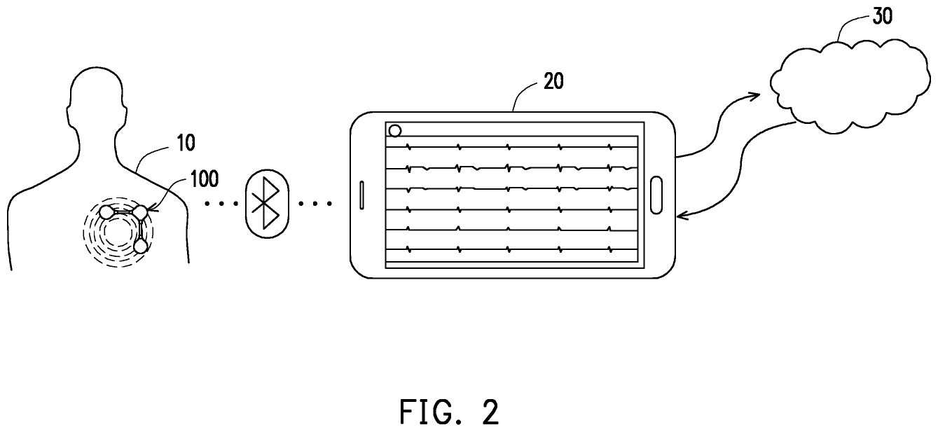

FIG. 2 is a schematic diagram of the electrocardiogram measuring device in FIG. 1 during measurement. With reference to FIG. 2, the electrocardiogram measuring device 100 is disposed on a human body 10. The Bluetooth chip 116 (FIG. 1) may be configured to transfer the signal measured by the electrocardiogram measuring device 100 in the wireless manner to an external electronic device 20, such as a mobile phone, or a computer. The electronic device 20 may subsequently transfer the measurement results to a cloud system 30. The cloud system 30 may have an AI interpretation system that may be configured to interpret the measurement results, and the cloud system 30 may then return the interpretation results to the electronic device 20. The AI interpretation system may continuously learn new measurement results and interpretation results to increase accuracy of interpretation.

With reference to FIG. 1 again, the gyroscope 117 may be configured to detect a movement state of the user and return the information back to the processor 115. The processor 115 may compensate for the noise caused by the movement of the user or ignore signal fragments with too much noise by calculations.

In addition, the artificial intelligence chip 118 is, for example, an AI edge computing chip. The designer may input learned AI calculations into the artificial intelligence chip 118. In this way, the electrocardiogram measuring device 100 may use the artificial intelligence chip 118 to interpret the measurement results by itself, without requiring a network connection to an external interpretation system.

In the embodiment, the electrocardiogram measuring device 100 may also include a warning device, such as a buzzer or a warning light. A warning may be directly issued to the user to remind him/her when the results is determined by the artificial intelligence chip 118 determines to be in need of warning. In an embodiment, the electrocardiogram measuring device 100 may also include a storage medium, such as a memory (flash), to record the measurement information.

It should be noted that in the embodiment, the non-zero included angle θ1 formed between the two connecting members 140 uses the example of 90 degrees, but it is not limited thereto. The non-zero included angle θ1 formed between the two connecting members 140 may be between 60 degrees and 120 degrees, and good measurement results would still be obtained.

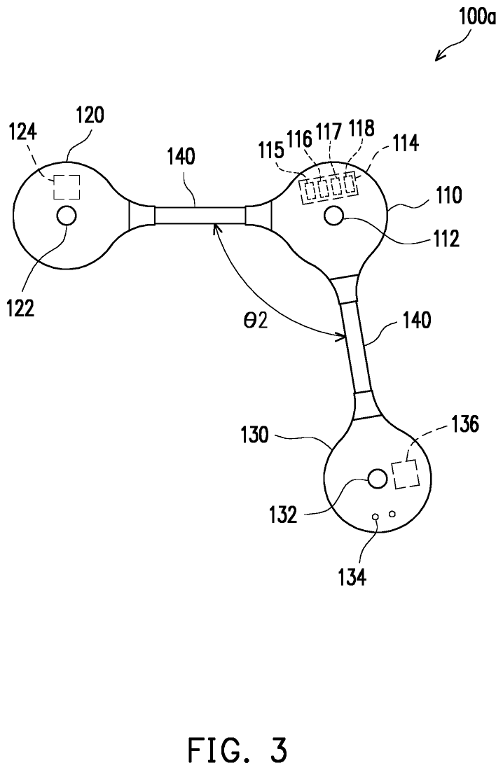

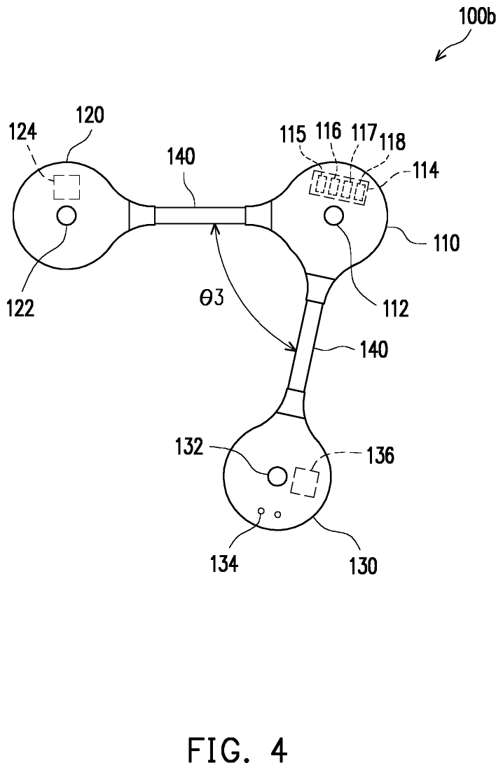

For example, FIG. 3 is a schematic diagram of an electrocardiogram measuring device according to another embodiment of the disclosure. With reference to FIG. 3, in the embodiment, a non-zero included angle θ2 formed between the two connecting members 140 of an electrocardiogram measuring device 100a is between 90 degrees and 120 degrees. FIG. 4 is a schematic diagram of an electrocardiogram measuring device according to yet another embodiment of the disclosure. With reference to FIG. 4, in the embodiment, a non-zero included angle θ3 formed between the two connecting members 140 of an electrocardiogram measuring device 100b is between 60 degrees and 90 degrees. Both the electrocardiogram measuring devices 100a and 100b shown in FIGS. 3 and 4 may have good measurement results.

In summary, the electrocardiogram measuring device according to the disclosure uses the two connecting members to fix the relative positions of the first machine body, the second machine body and the third machine body. The relative positions of the first electrode, the second electrode and the third electrode are fixed correspondingly too. Compared with the conventional electrocardiogram, in which the operator has to place the electrodes one by one, and the relative positions of the electrodes may be wrong, causing the measurement result to be distorted, the electrocardiogram measuring device according to the disclosure provides an alternative that can be operated by the general user by himself/herself, while being quite convenient to use.

Although the disclosure has been disclosed with the foregoing exemplary embodiments, it is not intended to limit the disclosure. Any person skilled in the art can make various changes and modifications within the spirit and scope of the disclosure. Accordingly, the scope of the disclosure is defined by the claims appended hereto and their equivalents.

What is claimed is:

1. An electrocardiogram measuring device, comprising:
a first machine body, having an exposed first electrode;
a second machine body, having an exposed second electrode;
a third machine body, having an exposed third electrode;
a circuit module, disposed in one of the first machine body, the second machine body, and the third machine body, wherein the first electrode, the second electrode, and the third electrode are connected to the circuit module via an electrical signal; and
two connecting members, wherein one of the connecting members is located between the first machine body and the second machine body, and is connected to the first machine body and the second machine body, and the other connecting member is located between the first machine body and the third machine body, and is connected to the first machine body and the third machine body, and a non-zero angle is formed between the two connecting members, wherein the first machine body, the second machine body, and the third machine body fix their relative positions through the two connecting members.

2. The electrocardiogram measuring device according to claim 1, wherein the two connecting members are two transmission lines, and the first machine body, the second machine body, and the third machine body are electrically connected to each other through the two transmission lines.

3. The electrocardiogram measuring device according to claim 1, wherein the non-zero included angle formed between the two connecting members is between 60 degrees and 120 degrees.

4. The electrocardiogram measuring device according to claim 1, wherein the non-zero included angle formed between the two connecting members is 90 degrees.

5. The electrocardiogram measuring device according to claim 1, wherein the two connecting members have a same length, which enables the second machine body and the third machine body to be symmetrically disposed on two sides of the first machine body.

6. The electrocardiogram measuring device according to claim 1, wherein the electrocardiogram measuring device further comprises at least one battery, which is disposed in at least one of the first machine body, the second machine body, and the third machine body.

7. The electrocardiogram measuring device according to claim 6, wherein the at least one battery comprises two batteries, which are disposed in the second machine body and the third machine body, and the circuit module is disposed in the first machine body.

8. The electrocardiogram measuring device according to claim 1, wherein the circuit module comprises a processor and a Bluetooth chip electrically connected to the processor, and the first electrode, the second electrode, and the third electrode are electrically connected to the processor.

9. The electrocardiogram measuring device according to claim 1, wherein the circuit module comprises a processor and a gyroscope electrically connected to the processor, and the first electrode, the second electrode, and the third electrode are electrically connected to the processor.

10. The electrocardiogram measuring device according to claim 1, wherein the circuit module comprises a processor and an artificial intelligence chip electrically connected to the processor, and the first electrode, the second electrode, and the third electrode are electrically connected to the processor.

11. The electrocardiogram measuring device according to claim 1, wherein length, width or diameter of any one of the first machine body, the second machine body, and the third machine body are respectively between 2 cm and 8 cm, and length of any one of the two connecting members is between 2 cm and 8 cm.
